(19) United States
(12) Patent Application Publication
(10) Pub. No.: US 2010/0120776 A1
(43) Pub. Date: May 13, 2010

(54) Carbocysteine medical foods

(75) Inventors: Jeffrey S. Kiel, Gainesville, GA (US); H. Greg Thomas, Carrollton, GA (US)

Correspondence Address: Collen A. Beard, Esq., 2225 Centennial Drive, Gainesville, GA, 30504, US

(73) Assignee: Hall Bioscience Corporation

(21) Appl. No.: 12/590,665

(22) Filed: Nov. 12, 2009

Related U.S. Application Data
(60) Provisional application No. 61/198,980, filed on Nov. 12, 2008.

(51) Int. Cl. A61K 31/197; C07C 321/04; A61K 31/519; A61P 39/06; A61P 11/00

(52) U.S. Cl. 514/249; 562/556; 514/562

(57) ABSTRACT

Medical foods containing carbocysteine and designed for buccal absorption; useful for treating conditions that benefit from administration of an antioxidant, immunostimulant, and/or mucoregulator, such as COPD.

### RELATED APPLICATION

The present application is related to and claims priority to U.S. Provisional Application Ser. No. 61/198,980, filed Nov. 12, 2008, the entire contents of which is incorporated herein by reference.

### FIELD OF THE INVENTION

The present invention relates to medical foods containing S-carboxymethyl-L-cysteine (carbocysteine). More specifically, the invention relates to carbocysteine medical foods designed for increased buccal absorption. The medical foods can be used, for example, as antioxidants and immunostimulants.

### BACKGROUND OF THE INVENTION

Carbocysteine is an antioxidant and thus may have an effect on many processes in the body. In particular, carbocysteine has been shown to be a scavenger of reactive oxygen intermediates (ROIs) and to reduce oxidative stress. Antioxidants are also useful for regulation of nitric oxide levels, the neutralization of free radicals, and maintenance of exogenous antioxidants such as vitamins C and E in their reduced (active) forms. Antioxidants are also widely used as nutritional supplements in the hope of maintaining health and preventing diseases such as cancer and coronary heart disease.

Carbocysteine is also known to act as an immunostimulant—a substance that increases the ability of the immune system to fight infection and disease. Carbocysteine has been shown to inhibit attachment of Haemophilus influenza, Streptococcus pneumoniae, and Moraxella catarrhalis to epithelial cells. Carbocysteine has antiviral activity as well, having been shown to inhibit rhinovirus infection.

Carbocysteine is also a mucoregulating agent. It lowers the viscosity and increases the volume of mucus, thereby facilitating expectoration. Carbocysteine has been used for many years in the treatment of conditions characterized by excessive viscous mucus such as chronic obstructive pulmonary disease (COPD) and otitis media with effusion (“glue ear”). Mucoregulators are also useful in chest problems such as bronchitis, asthma, and cystic fibrosis.

Carbocysteine is known to be well tolerated and is generally administered orally, either in solid dosage form such as tablets or capsules or in the form of syrups. Carbocysteine has also been used as an anti-infective nasal spray with mucoregulating and expectorant action.

Carbocysteine is sold in the United Kingdom by prescription as Mucodyne. It is available as a syrup, a pediatric syrup, and capsules and is sold as a “mucolytic, an agent that causes the phlegm to be less sticky and easier to cough up”. Methylcysteine, an L-cysteine analog, is available as Visclair, a prescription mucolytic, in the United Kingdom. It is an oral tablet. Another cysteine analog is N-acetylcysteine, which is available by prescription as Mucomyst and Mucocil, delivered as a liquid inhalant (as a mucolytic agent) or as a liquid for intravenous administration (as an acetaminophen antidote). N-acetylcysteine is also available as a non-prescription nutritional supplement in the United States, marketed as an antioxidant. N-acetylcysteine is more stable than cysteine but presents taste masking issues due to its free sulfur group and may have toxicity issues due to its acetyl group. In addition, direct mucolytics such as N-acetylcysteine, must be employed in high concentrations to achieve the desired effect, which increases the risk of undesirable side effects.

Traditional oral administration of carbocysteine by solid form is inappropriate in some cases for several reasons. Traditional tablets and capsules are inconvenient or impractical for patients who have trouble swallowing these dosage forms, such as elderly people and young children. Patients undergoing radiation therapy or chemotherapy may be too nauseous to swallow tablets and capsules. Patients with neurological disorders or who are mentally ill may hide tablets under the tongue to avoid swallowing the medication. In addition, water needed to wash down a tablet or capsule may not be available or it may be desirable to avoid fluids, such as prior to bedtime. In addition, in cases where the patient has a sore throat or mucus buildup, swallowing may be difficult.

Moreover, oral administration of solid medications has disadvantages such as hepatic first pass metabolism and enzymatic degradation within the GI tract. This prohibits oral administration of certain classes of drugs especially peptides and proteins. Consequently, other absorptive mucosas are considered as potential sites for drug administration. Transmucosal routes of drug delivery (i.e., the mucosal linings of the nasal, rectal, vaginal, ocular, and oral cavity) offer distinct advantages over oral administration for systemic drug delivery. These advantages include possible bypass of first pass effect, avoidance of presystemic elimination within the GI tract, and, depending on the particular drug, a better enzymatic flora for drug absorption.

Cysteines (methylcysteine, carbocysteine, n-acetylcysteine, L-cysteine) are known to have low bioavailablity, due to several reasons. Cysteines are unstable and may decompose due to low pH, high temperature, and oxidation. Some cysteines, such as L-cysteine, may not reach the intestines since they decompose in the stomach. Absorption may also be limited by food and intestinal conditions.

Consequently, there is a need for a carbocysteine formulation that releases the carbocysteine in the oral cavity, avoiding the need for swallowing. There is a need for a carbocysteine formulation designed for buccal absorption.

Medical foods are products that are specially formulated and intended for the dietary management of a disease that has distinct nutritional needs that cannot be met by normal diet alone. Medical foods are defined in the Food and Drug Administration's 1988 Orphan Drug Act Amendments and are subject to the general food and safety labeling requirements of the Federal Food, Drug, and Cosmetic Act. Medical foods are distinct from the broader category of foods for special dietary use and from traditional foods that bear a health claim. In order to be considered a medical food the product must, at a minimum:

be a food for oral ingestion or tube feeding (nasogastric tube);

be labeled for the dietary management of a specific medical disorder, disease or condition for which there are distinctive nutritional requirements; and

be intended to be used under medical supervision.

Medical food products are often used in hospitals (e.g., for burn victims or kidney dialysis patients) and outside of a hospital setting under a physician's care (e.g., for PKU, AIDS patients, cardiovascular disease, osteoporosis) for the dietary management of diseases in patients with particular medical or metabolic needs due to their disease or condition.

It would be advantageous to have a medical food supplying carbocysteine. It would be advantageous to have a medical food containing carbocysteine which allows for buccal absorption of the carbocysteine.

### SUMMARY OF THE INVENTION

Various embodiments of the present invention provide for medical foods containing carbocysteine in a desired dosage amount. Desirably the medical foods are in a dosage form designed to release carbocysteine in the buccal cavity and promote buccal absorption. Methods for the dietary management of oxidative stress are provided.

While the medical foods containing carbocysteine are described herein primarily for use as an antioxidant, it should be understood that they could also be used for other purposes such as an immunostimulant or mucoregulator. The medical foods could be used as nutritional support for those having COPD, in which its antioxidant, immunostimulatory, and mucoregulating activities could provide benefits. Another preferred application for the medical food is nutritional support for people afflicted with asthma.

Accordingly, it is an object of the invention to provide medical foods containing carbocysteine. These medical foods desirably deliver carbocysteine for buccal absorption, although they could be designed for other types of administration. The medical foods can be in the form of chewable tablets, fast dissolving tablets, or other buccal dosage forms. The medical foods can contain other ingredients such as additional active ingredients (i.e., additional antioxidants), absorption enhancers, taste enhancers, and conventional formulation additives.

It is also an object of the invention to provide medical foods for the dietary management of pulmonary oxidative stress. Examples of conditions that could benefit from the medical foods are COPD and asthma.

### DETAILED DESCRIPTION OF THE INVENTION

It should be understood that buccal absorption as used herein refers to absorption through any oral cavity mucosa, including the buccal mucosa (mucosal membranes lining the cheeks) as well as sublingual mucosa (mucosal membranes lining the floor of the mouth). Buccal absorption means that the active agent is absorbed primarily buccally, at least more so than in oral administration dosage forms.

Dosage Form

The medical food dosage form can be a liquid or solid. In a preferred embodiment, the medical food is supplied as a dosage form that releases the carbocysteine in the oral cavity-termed a buccal delivery dosage form herein- and allows for buccal absorption. Examples include fast dissolving tablets, chewable tablets, lozenges, gels, wafers, microparticles, chewing gums, patches, films, sprays, and mouthwashes.

As used herein, a fast dissolving tablet is defined as a tablet that disintegrates and/or dissolves rapidly in the saliva without the need for water. Fast dissolving tablets include tablets that dissolve in saliva within a few seconds as well as those which contain agents to enhance the rate of tablet disintegration in the oral cavity and take up to a few minutes to completely disintegrate (sometimes called fast disintegrating tablets). When placed in the mouth, the fast dissolving tablet releases the carbocysteine, which is absorbed through the mucosal lining. As used herein, a chewable tablet is defined as a tablet designed to be masticated to speed disintegration and dissolution. The term buccal tablet as used herein includes fast dissolving tablets, fast disintegrating tablets, and chewable tablets. A buccal tablet can dissolve in a few seconds, take as long as a few minutes, or be masticated to aid in drug release.

In one embodiment, the medical food is a fast dissolving tablet which is designed to be taken twice a day. Each tablet contains 750 mg carbocysteine in a fast melt formulation. In another embodiment, the medical food is a chewable tablet which is designed to be taken twice a day. Each tablet contains 750 mg carbocysteine. In another embodiment, the medical food is a liquid which is designed to be taken by mouth twice a day. Each teaspoonful (5 mL) contains 250 mg of carbocysteine. In a third embodiment, the medical food is a liquid designed to be sprayed into the mouth. Each metered dose spray (0.25 mL) contains 100 mg carbocysteine.

Other dosage forms are possible and the invention is not limited to any particular form. The dosing frequency and carbocysteine concentration can also be different from that described. Formulation of the medical foods will depend upon the indication for which the medical food is to be used and the dosing requirements. Examples of formulations are provided below and persons skilled in the art can devise other appropriate formulations.

Indications

The medical foods act as antioxidants and can be used for dietary management of diseases in which treatment with an antioxidant is helpful. The medical foods can also be used as immunostimulants or mucoregulating agents.

The medical foods may have an effect on many processes in the body, including regulation of nitric oxide levels, protection from oxidative stress, the neutralization of free radicals and reactive oxygen compounds, and maintenance of exogenous antioxidants such as vitamins C and E in their reduced (active) forms. The antioxidant activity of carbocysteine may have an additive effect to its mucoregulating activity with COPD and other diseases.

The medical foods containing carbocysteine can be used to reduce the incidence of the common cold and the incidence of respiratory infections by bacteria, due to the ability of carbocysteine to inhibit attachment of Haemophilus influenza, Streptococcus pneumoniae, and Moraxella catarrhalis to epithelial cells. Carbocysteine has antiviral activity as well, having been shown to inhibit rhinovirus infection.

The medical foods may be beneficial for use in diseases where excessive or overly thick mucus is an issue. Examples include chronic obstructive pulmonary disease (COPD), otitis media with effusion (“glue ear”), acute and chronic bronchitis, bronchiectasis, bronchorrhea, and sinusitis. People with asthma and other reactive airway diseases may benefit from consumption of the medical foods.

Although the medical foods are described for use in humans, they may also be appropriate for non-human animal use. Many animals suffer from excessive or overly thick mucus and might find relief from the medical foods. Of course, the dosage forms and amounts would vary for each species.

Other Ingredients

Common additives to buccal dosage forms include sweeteners, flavors, and taste maskers. Other ingredients include those commonly used to formulate tablets. It may be desirable to include enhancers to molecule permeation in the formulation. Such enhancers are known in the art and include various chelators, surfactants, bile salts, fatty acids, and chitosan.

Depending upon the disease to be treated, it may be desirable to include other active agents in the medical food. For example, other antioxidants may be helpful such as glutathione, vitamin C, and vitamin E. Still other additives include cofactors such as selenium, and other vitamins and minerals. Nutritional supplements might be useful in some formulations.

In one embodiment, folic acid is included. Anemia has been found to occur in many COPD patients and folic acid is commonly used for treatment of anemia. It can be included in amounts known in the art, or from about 200 μg to 500 mg.

In another embodiment, another cysteine derivative is included that acts via a different pathway. For example, it is not believed that carbocysteine adds directly to the levels of cysteine in the body. It may be useful to include a cysteine derivative that breaks down into cysteine, thus directly elevating the cysteine levels. Known cysteine derivatives that act in this manner include N-acetylcysteine and N, N′-diacetyl-L-cystine and they could be included from about 150 to 2000 mg.

Methods of Use

Preferably, the medical foods of the present invention are administered two or three times per day, preferably at least once in the morning and once in the evening. A typical treatment regime for the medical foods will continue for several weeks and may be required for up to six months to one year or more. Depending on such factors as the reason for the treatment and the patient's response, the treatment regime may be extended.

Dosage size can range from 10 mg to 4000 mg, preferably about 100 mg to 2500 mg, more preferably about 750 mg per unit, although this will vary depending on the patient's size and physiology and the condition being treated. Of course, non-human animals may require different dosing.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### Example 1

### Carbocysteine Dissolving Tablets

1200 mg dissolving tablets containing 500 mg carbocysteine were made according to the following description. The tablets were made from the following composition.

| | Percent in tablet | Total batch weight (kg) |
|---|---|---|
| Ingredient | (w/w) | (see note 1) |
| Carbocysteine | 41.670% | 50.004 (55.004) |
| Povidone, K-30 | 3.000% | 3.600 (3.960) |
| F-Melt C (see note 3) | 43.83% | 52.596 (53.796) |
| Progeletinized Corn Starch, | 1.500% | 1.800 (1.980) |
| NF | | |
| Explotab ® (see note 4) | 4.000% | 4.800 |
| Sucralose, NF | 1.000% | 1.200 |
| Artificial Orange Flavor | 2.000% | 2.400 |
| Powder | | |
| Magnesium Stearate, NF | 2.000% | 2.400 |
| Talc | 1.0% | 1.200 |
| Purified Water | N/A | 20.000 (see note 2) |
| Total | | 120.00 kg |

1 The amount in parentheses indicates the amount added as overage in the wet granulation to account for losses.
2 Removed by drying.
3 F-Melt C ™ is a proprietary formulation of carbohydrates, disintegrants and inorganic ingredients made by Fuji Chemical Industry Co., Ltd., Japan. It includes 65% d-mannitol, 5% xylitol, 18% microcrystalline cellulose, and 8% crospovidone.
4 Explotab ® is a disintegrant made by JRS Pharma which is a mixture of sodium starch glycolate and sodium carboxymethyl starch.

### Procedure:

The carbocysteine, kollidon-30, pregeletinized corn starch and F-Melt C (13.200 kg) were weighed to a 2 cu. ft. paddle blender bowl. The powders were mixed for 2 minutes. Powder (1.5 kg) was drained from the outlet of the blender, and transferred back to the granulation. The powder was mixed for an additional 3 minutes. Water was sprayed on to the powders in the blender and mixed for 10 minutes. The sides and the bottom of the blender were scraped, and mixed for an additional 3 minutes. The wet powder was transferred to perforated drying trays and spread evenly to a depth of no more than ½ inch.

The wet granulation powder was dried in a forced air oven at 60° C. until the moisture content was approximately 1.7%.

The dried granulation powder was milled using a 0.062″ screen size in a Fitz Mill for 3 minutes. The milled material was transferred to the 2 cu. ft. paddle blender and accurately weighed F-Melt C (40.596 kg), talc, sucralose, and orange flavor were added. The powder was mixed for 2 minutes. The sides and bottom of the blender were scraped, then mixed for an additional 10 minutes. Accurately pre-weighed magnesium stearate was added to the powder and mixed for 2 minutes. The resulting dry blend powder was used for tableting.

A Manesty-16 station Tablet press, and round punches were used to compress the dry blend into 1200 mg tablets. The average tablet weight was 1212 mg and the average hardness was 7.2 kp.

### Example 2

### Carbocysteine Dissolving Tablets

850 mg dissolving tablets containing 400 mg carbocysteine were made according to the formulation. The procedure was similar to that of Example 1.

| | Percent in tablet | Total weight in tablet |
|---|---|---|
| Ingredient | (w/w) | (mg) |
| Carbocysteine | 47.0588 | 400.00 |
| F-Melt | 33.1412 | 281.70 |
| Avicel microcrystalline | 15.0000 | 127.50 |
| cellulose | | |
| Corn Starch | 1.0000 | 8.50 |
| Sucralose | 1.0000 | 8.50 |
| Magnesium Stearate, NF | 1.4000 | 11.90 |
| Talc | 1.4000 | 11.90 |
| Total | | 850.0 |

### Procedure:

All ingredients except the magnesium stearate were mixed for 30 minutes in a planetary mixer. The magnesium stearate was added and the ingredients mixed for an additional two minutes. A BB2-27 tablet press was used for direct compression of the mixture into an 850 mg tablet.

### Example 3

### Buccal Availability Study

A study was conducted in dogs to determine the pharmacokinetics and bioavailability of a buccal formulation of carbocysteine in comparison to oral and IV routes of administration.

The study was conducted according to the following methodology. A 400 mg dissolving tablet, a 400 mg oral tablet, and a 200 mg IV dose of carbocysteine were administered to each of five beagle dogs in a randomized crossover pattern. Both the 400 mg dissolving tablet and the 400 mg oral tablet used in the study were prepared according to Example 2. The dissolving tablet was administered to the animal by placing the tablet in the mouth until it dissolved. The oral tablet was administered by forcing the animal to swallow the tablet. Plasma samples were collected at 0, 5, 10, 20, 30, 45, 60, 75, 90, 105, 120, 150, 180, 270, 360, and 450 minutes. Plasma concentrations were measured by precolumn derivatization of S-carboxymethylcysteine using o-phthaldialdehyde (OPA) and subsequent HPLC analysis. All data were analyzed using WinNonlin®, a tool for pharmacokinetic and pharmacodynamic analysis. The carbocysteine plasma data was fitted to a one-compartment model with first-order elimination. The pharmacokinetic parameters (t_{1/2}, CL, Vd) generated from each route of administration were compared using analysis of variance (ANOVA) to detect statistically significant differences. A paired t-test was used to detect the differences in Cmax, Tmax, Ka, and F between buccal and oral routes of administration.

It was found that there were no significant differences in t_{1/2} (61−64 min), Vd (405−418 ml/kg) and CL (4.52−4.64 ml/min/kg) among the different routes of administration. In addition there were no significant differences in Ka (0.028−0.034 min⁻¹) and Tmax (90−93 min) between buccal and oral routes of administration. But the absolute bioavailability F and Cmax of buccal formulation (83.2±8.4% and 43.4±8.0 mg/L) were significantly higher than those of oral formulation (51±1.4% and 29.5±8.0 mg/L). It can be concluded that the buccal formulation exhibited a two-fold increase in bioavailability as compared to the oral tablet. This increase observed with the buccal formulation results primarily from the decrease in first pass metabolism, which is extensive with oral formulations.

### Example 4

### Soy Based Disintegrant

1500 mg dissolving tablets containing 750 mg carbocysteine were made according to the following formulation. Encosoy®, offered by Mendel, consists of soy polysaccharides and a non-ionic effective disintegrant. A very similar procedure as Example 1 was used, with the exception of substitution of the Encosoy for Explotab as the disintegrant.

| | Percent in tablet | Total weight in tablet |
|---|---|---|
| Ingredient | w/w | (mg) |
| Carbocysteine | 50.00 | 750.00 |
| Kollidon 30 | 4.00 | 60.00 |
| F-Melt C | 34.5 | 517.50 |
| Progeletinized Corn Starch, | 1.5 | 22.50 |
| NF | | |
| Encosoy ® | 4.00 | 60.00 |
| Sucralose, NF | 1.00 | 15.00 |
| Artificial Orange Flavor | 2.00 | 30.00 |
| Powder | | |
| Magnesium Stearate, NF | 2.00 | 30.00 |
| Talc | 1.00 | 15.00 |
| Purified Water | NA | 50.00 |
| Total | | 1500.00 |

### Example 5

### Oral Solution

| | Percent in solution | Total weight in 500 ml |
|---|---|---|
| Raw Material | (w/w) | unit (g) |
| Carbocysteine | 5.00 | 25.0 |
| Sodium tetraborate, | 9.534 | 47.67 |
| decahydrate | | |
| Sucralose, NF | 0.2 | 1.00 |
| Artificial Orange Flavor | 0.2 | 1.00 |
| Powder | | |
| Sodium methylparaben | 0.2 | 1.00 |
| Purified Water | | q.s. to 500 ml |
| Total | | 500 ml |

The ingredients were added individually to 400 ml purified water and mixed until dissolved. pH was adjusted to 7.5 and q.s. with purified water to 500 ml.

### Example 6

### 250.00 mg/ml Suspension

| | Percent in suspension | Total batch weight |
|---|---|---|
| Raw Material | (w/w) | (g) |
| Carbocysteine | 25.000 | 5000.00 |
| Xanthan gum | 0.400 | 80.00 |
| Cherry bubblegum | 0.250 | 50.00 |
| (arylessence) | | |
| Glycerin | 74.150 | 14830.00 |
| Sodium methylparaben | 0.200 | 40.00 |
| Total | | 20000.00 ml |

The ingredients were added in the following order to a mixing tank, mixing after each addition until dissolved: 8000 ml glycerin, xanthan gum, carbocysteine, cherry bubblegum flavor, sodium methylparaben. The remainder of the glycerin was added and the suspension mixed an additional 30 minutes.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

1. A medical food comprising carbocysteine in a dosage form for buccal absorption.

2. The medical food of claim 1, wherein the medical food is administered as an antioxidant, immunostimulant, or mucoregulator.

3. The medical food of claim 1, wherein the medical food is administered to patients having a disease selected from the group consisting of chronic obstructive pulmonary disease (COPD), otitis media with effusion (“glue ear”), acute and chronic bronchitis, bronchiectasis, bronchorrhea, sinusitis, and asthma.

4. The medical food of claim 1, designed to treat chronic obstructive pulmonary disease (COPD).

5. The medical food of claim 1, formulated as a fast dissolving tablet or chewable tablet.

6. The medical food of claim 1, further comprising folic acid.

7. The medical food of claim 1, further comprising a cysteine derivative.

8. A method for delivering an antioxidant comprising administering a medical food containing carbocysteine in a buccal absorption dosage form.

9. The method of claim 8, wherein the medical food is a fast dissolving tablet or chewable tablet.

10. A method for treating a disease where excessive or overly thick mucus is an issue comprising administering a medical food containing carbocysteine in a buccal absorption dosage form.

11. The method of claim 10 wherein the disease is selected from the group chronic obstructive pulmonary disease (COPD), otitis media with effusion (“glue ear”), acute and chronic bronchitis, bronchiectasis, bronchorrhea, sinusitis, and asthma.

12. The method of claim 10, wherein the medical food is a fast dissolving tablet or chewable tablet.
